(19) United States
(12) Patent Application Publication
(10) Pub. No.: US 2008/0077116 A1
(43) Pub. Date: Mar. 27, 2008

(54) CONTAINER FOR INTRAVENOUS FLUIDS

(76) Inventors: Rosemary Dailey, Lexington, KY (US); Joyce Corbin, Lexington, KY (US)

Correspondence Address: KING & SCHICKLI, PLLC, 247 NORTH BROADWAY, LEXINGTON, KY, 40507, US

(21) Appl. No.: 11/854,144

(22) Filed: Sep. 12, 2007

Related U.S. Application Data
(60) Provisional application No. 60/843,861, filed on Sep. 12, 2006.

(51) Int. Cl. A61M 5/14

(52) U.S. Cl. 604/410

(57) ABSTRACT

A container includes one or more first and second portions and at least one divider for providing access to the second portion from an interior of the first portion, and for preventing admixing of the contents of the first and second portions. A length of tubing defining a lumen may be provided, adapted to place the lumen in fluid communication with the first and second portions. Still further, a method for delivering a medication is provided, including the steps of providing a container and tubing as described, placing the tubing lumen in fluid communication with the container first portion and passing at least a sufficient volume of the purge fluid through the lumen to substantially purge air or other fluids. Next, the lumen is placed in fluid communication with the at least one second portion to deliver a volume of the medication into the blood vessel.

This application claims the benefit of priority in U.S. Provisional Patent Application Ser. No. 60/843,861, filed on Sep. 12, 2006, the disclosure of which is incorporated herein in its entirety by reference.

### TECHNICAL FIELD

The present invention relates to containers for holding fluid destined for delivery to a blood vessel of an individual. In particular, the invention relates to a container including a separate portion for holding a purge fluid, to allow purging of air from the lumen of tubing such as intravenous tubing, whereby the fluid for delivery to the blood vessel and the purge fluid are not admixed. A method for purging air from the lumen of an intravenous line prior to introduction of a fluid into the vein of a patient in need of such intravenous fluid is provided also.

### BACKGROUND OF THE INVENTION

It is well known to provide a sterile, sealed container for holding sterilized fluids such as intravenous fluids, for eventual delivery into a blood vessel of an individual in need of such fluids. This is a common means of delivery of various medications, electrolyte solutions, saline solutions, and the like. Provision of sealed containers already charged with a desired fluid allows maintaining the fluid under aseptic conditions. Still further, pre-packaging of such fluids affords convenience in transport, storage, and shipping. To deliver the fluid from a sealed container to the individual's blood vessel, it is known also to provide sterile tubing. One end of the tubing may be placed in fluid communication with the fluid to be delivered, such as by puncturing a septum or a port of known design with a hollow needle or trocar connected to the tubing, and the other end may be introduced into a blood vessel of the individual through the skin, such as by a similar needle, trocar, or catheter.

However, generally such intravenous tubings or lines are delivered initially containing a volume of a fluid such as air or other gases, substantially equivalent to the internal volume of the tubing lumen. Simply introducing the needle into the individual's blood vessel and beginning delivery of the intravenous fluid would therefore also deliver the volume of air into the blood vessel. This can result in a condition known as air embolism or gas embolism, which results from the introduction of gas bubbles in the bloodstream. Depending on the amount of air or other gas introduced and where the bubbles lodge in the individual's body, air embolism can cause symptoms of varying severity, including skin rash, numbness, visual disturbances, shortness of breath, paralysis, and cardiac arrest.

It is therefore common practice in the medical and veterinary arts, when delivering an intravenous fluid to a patient, to allow a sufficient quantity of the fluid to pass through the intravenous tubing to displace the volume of air contained therein prior to introducing the intravenous fluid into the individual's vein. Depending on the length and inner diameter of the tubing, this practice may result in wasting as much as 5-30 ml or more of the intravenous fluid.

While this amount may seem insignificant, 5-30 ml of a particularly expensive medication may represent a significant financial impact in and of itself. Further, often in treating a disease it is deemed essential to provide a specified amount of a particular medication during a particular treatment session, which is provided in the volume included in the container. Wasting 5-30 ml of the intravenous medication in the first container may require using a portion of a second container of medication to deliver the required dose to the patient. The unused portion of the second container can generally not be given to a different patient, due to issues of disease transfer and/or the finite shelf life of many medications. The cost issue of wasting a particularly expensive medication is thus exacerbated. This cost must then be passed on to the patient or the patient's health care insurance provider, adding to the already burdensome cost of medical care and treatment.

There is accordingly a need in the art for methods for delivering fluids such as intravenous fluids, and for containers for holding a fluid destined for intravenous use, wherein such waste is reduced or eliminated. Desirably, only a single container should be required, and there should be no transfer of needles or trocars after the initial puncture of the container, thus reducing the risk of compromising the required sterile conditions while delivering intravenous fluids to the patient.

In accordance with this need, the present invention provides a container including a first portion for use in purging air or gas from a lumen of an intravenous tubing, and including also a second portion for holding a second fluid for holding a fluid containing a medication or other solution of choice. The present invention also provides a method for delivering an intravenous fluid or solution into a blood vessel of an individual in need of such fluid or solution, whereby air or other gas is purged from an intravenous line without wasting any portion of the intravenous fluid.

### SUMMARY OF THE INVENTION

In one aspect, the present invention provides a container for holding an intravenous fluid, comprising at least one first portion for holding at least one first or purging fluid for purging air or other gas from a lumen of a tubing and at least one second portion for holding at least one second fluid which may be a medication, such as a fluid drug, an electrolyte solution, a saline solution, or the like. The container includes at least one divider for separating the purge fluid and medication, and for providing access to an interior of the at least one second portion from an interior of the at least one first portion. The at least one divider further prevents substantial admixing of the purge fluid and medication, including when the interior of the at least one second portion is accessed from the interior of the first portion.

Typically, the at least one divider includes at least one self-sealing access point between the at least one first portion and the at least one second portion. The at least one self-sealing access point may be fabricated from a suitable self-sealing material. In one embodiment, the at least one self-sealing access point is at least one divider-traversing port having a self-sealing septum. Alternatively, the at least one divider itself may include at least a portion manufactured of a suitable self-sealing material. The at least one first portion of the container may be spatially oriented below the at least one second portion, with the divider interposed therebetween.

In another aspect of the present invention there is provided an assembly for holding and delivering an intravenous fluid, comprising a container as described above. That is, the container comprises at least one first portion for holding a purge fluid and at least one second portion for holding a medication.

The container further comprises at least one divider for separating the purge fluid and the medication whereby the purge fluid and the medication are not substantially admixed, including when an interior of the at least one second portion is accessed from an interior of the first portion. The at least one divider may be substantially as described above.

The assembly further includes a length of tubing defining a lumen, wherein the tubing is adapted to place the lumen in fluid communication with the at least one first portion and the second portion of the container. Typically, the at least one first portion of the container holds a volume of fluid at least sufficient to substantially purge air or other fluid from a lumen of the tubing, and may be adapted to hold only a sufficient volume to purge that lumen. The at least one first portion may be spatially oriented beneath the at least one second portion to allow optimal gravity-feed of the contents thereof, although alternative spatial arrangements are possible, such as for delivery of the container contents using a peristaltic pump or the like.

In yet another aspect of the present invention, a method is provided for delivering a medication into a blood vessel of an individual in need of such medication. The method of the present invention comprises the steps of providing a container substantially as described above, having at least one first portion containing a purge fluid and at least one second portion containing a medication. For the present method, the container comprises at least one divider as described above, which prevents substantial admixing of the purge fluid and the medication, including when an interior of the at least one second portion is accessed from an interior of the first portion. The at least one first portion may be spatially oriented below the at least one second portion with the at least one divider interposed therebetween, for optimal gravity dispensing of the contents of the container. The at least one first portion may be adapted to hold only a sufficient volume of purge fluid to purge the tubing lumen of air or other gases.

A length of tubing defining a lumen is also provided, followed by the steps of placing the tubing lumen in fluid communication with the at least one first portion and passing at least a sufficient volume of the purge fluid through the lumen to substantially purge air or other fluids, and then placing the lumen in fluid communication with the at least one second portion to deliver a volume of the medication.

A hollow needle, cannula, or trocar may be provided an end of the tubing, such as at opposed tubing ends, for piercing the first and/or second portions of the container and delivering the medication into the blood vessel, respectively. The needle or trocar may be advanced into the at least one first portion of the container, and a sufficient volume of purge fluid allowed to pass through the tubing lumen whereby the volume of fluid contained therein is substantially displaced. The needle or trocar may then be advanced into the second portion through the at least one divider for delivery of the medication.

### BRIEF DESCRIPTION OF THE DRAWING

The accompanying drawings incorporated in and forming a part of the specification, illustrates several aspects of the present invention, and together with the description serves to explain the principles of the invention. In the drawings:

FIG. 1 depicts a container in accordance with the present invention;

FIG. 2 depicts a container in accordance with another embodiment of the present invention;

FIG. 3 depicts a container in accordance with yet another embodiment of the present invention;

FIGS. 4a-4c depict a method for using the container of FIG. 1;

FIGS. 5a-5b depict additional embodiments of the present invention;

FIG. 6 depicts a container in accordance with yet another embodiment of the present invention, having multiple first portions; and

FIG. 7 depicts a container in accordance with yet another embodiment of the present invention, having multiple first and second portions.

### DETAILED DESCRIPTION OF THE INVENTION

In accordance with the foregoing need identified in the art, in one aspect the present invention provides a container 10 for holding a fluid such as for intravenous delivery. The container 10 comprises at least one first portion 12 for holding at least one first fluid 13 such as a purge fluid, and at least one second portion 14 for holding a second fluid 15 such as a medication, that may be different from the first fluid 13, wherein the first and second fluids 13, 15 are not admixed. Advantageously, the container 10 of the present invention allows delivery of an inexpensive purging fluid to remove air or other gases from the lumen of intravenous tubing, followed by delivery of a predetermined amount of an intravenous fluid, all from the same container and all without wasting costly intravenous fluid.

It will be appreciated by the skilled artisan that the container 10 may take the form of any of a number of containers known in this art, such as a rigid-sided bottle (not shown), a semi-rigid-sided bottle (not shown), or a flexible pouch or bag such as is depicted the Figures presented herein. Still further, the container 10 may be fabricated of (or include portions or components fabricated of) any suitable materials as are known in the art for preparing containers for delivering intravenous fluids, such as various polymers or plastics, polyolefin, ethylene vinyl acetate, polyvinylchloride, polypropylene, vinyl, polystyrene, glass, silicone glass, laminated aluminum foil, polyester, and combinations thereof. The container 10 may be transparent, translucent, or opaque in accordance with the light-sensitivity of the compositions to be held therein, and/or with the user's need or desire to visualize the contents of the container 10. For example, the user may wish to verify that no portion of a particular medicament has precipitated from its carrier or diluent, or alternatively after having introduced a medicament into a diluent held within container 10 may desire to verify that the two have completely mixed.

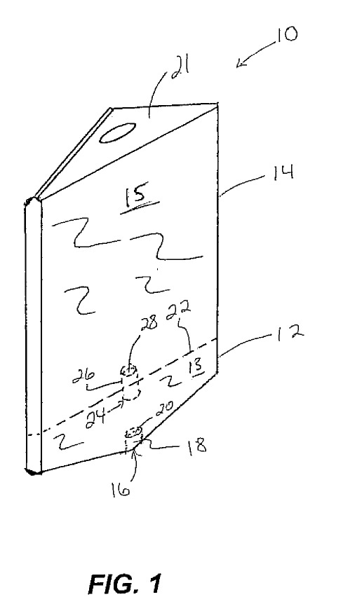

In the embodiment depicted in FIG. 1, the container 10 includes an external access point 16, which may be a port 18 having an internal self-sealing septum 20 of a design known in the art. The septum 20 may be fabricated of any suitable elastomer, such as rubber, siliconized rubber, or the like which may be perforated by a needle or the like, and which is known to substantially seal itself after withdrawal of such a needle. It is known in this art to deliver a fluid from containers such as intravenous bags using a gravity-feed. Accordingly, as shown in FIG. 1, access point 16 may be positioned at a bottom-most end of container 10 to ensure full delivery of the contents of container 10. Optionally, a hanger portion 21 may be provided at an end of container 10 opposite access point 16, to conveniently allow hanging container 10 from a hook or an i.v. stand (not shown). Hanger portion 21 may be an apertured flange such as is depicted in FIG. 1, or may be as simple as a cord, wire, hook, or the like (not shown) attached to a top edge of container 10.

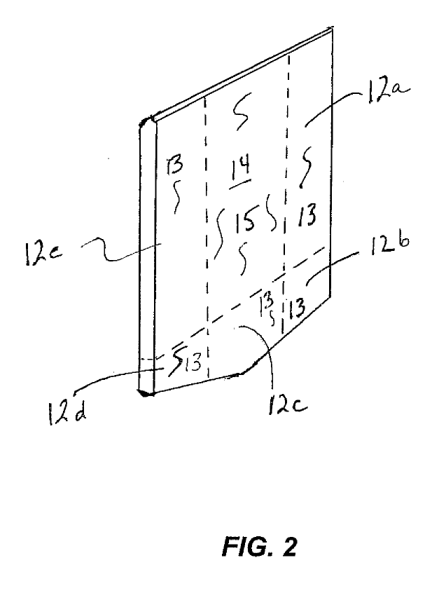

However, it is also known to deliver the contents of containers such as intravenous bags using devices such as peristaltic pumps or infusion pumps (not shown). Such peristaltic pumps or infusion pumps allow more precise delivery of specific volumes of fluid over a predetermined period of time. Accordingly it will be appreciated that first portion 12 and access point 16 need not be positioned at the bottom-most end of container 10 as would be optimal for a gravity-feed system. Indeed, one or more first portions 12 (see 12a-12e in FIG. 2) may be provided, positioned at any desired location on or within container 10, for use with an infusion pump or peristaltic pump, without concern for the need for a gravity-feed to ensure complete delivery of the contents of container 10. The only provisos are that admixing between the contents of first portions 12 and second portions 14 must be prevented.

The first portion 12 may be separated from the second portion 14 by a divider 22. In the embodiment shown in FIG. 1, first portion 12 is spatially oriented below second portion 14, in a vertically stacked arrangement with divider 22 interposed therebetween, allowing a gravity feed of the contents of first and second portions 12, 14. It will be appreciated that first portion 12 should hold a volume of first fluid 13, which may be an inexpensive purging fluid, at least sufficient to remove air or other gases from the lumen of intravenous tubing as described above. In one embodiment of the present invention, first portion 12 holds only a sufficient volume of fluid 13 to purge the tubing lumen, such as in the case where only a single use of container 10 is to be made.

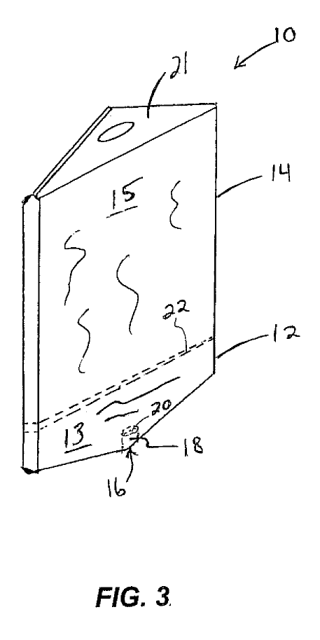

Divider 22 may be as simple as a seal or crimp traversing a section of container 10, whereby the contents of first portion 12 and second portion 14 are separated such that they do not admix. At least one internal access point 24 is provided between the first portion 12 and the second portion 14. In the embodiment depicted in FIG. 1, access point 24 may be a second, divider-traversing port 26, having a self-sealing septum 28 as described above. Alternatively, divider 22 may simply be a partition or wall dividing container 10 into a first portion 12 and a second portion 14 (see FIG. 3), without a discrete access point 16 as shown in FIG. 1. In this embodiment, at least a portion of divider 22 is preferably fabricated of a self-sealing material as described above, whereby the contents of first portion 12 and second portion 14 are kept separated even if an interior of second portion 14 is accessed from an interior of first portion 12. That is, in this embodiment at least a portion of divider 22 may be fabricated of a suitable self-sealing material such that the divider 22 itself functions as a self-sealing septum interposed between first portion 12 and second portion 14.

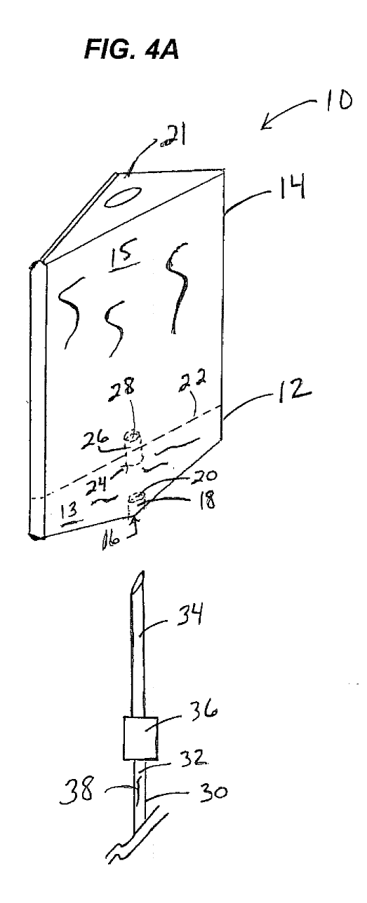

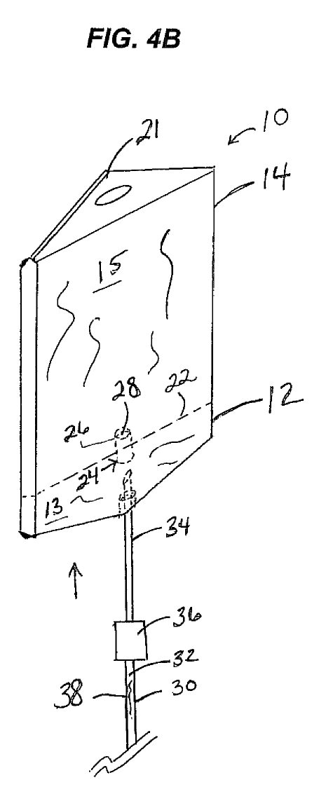

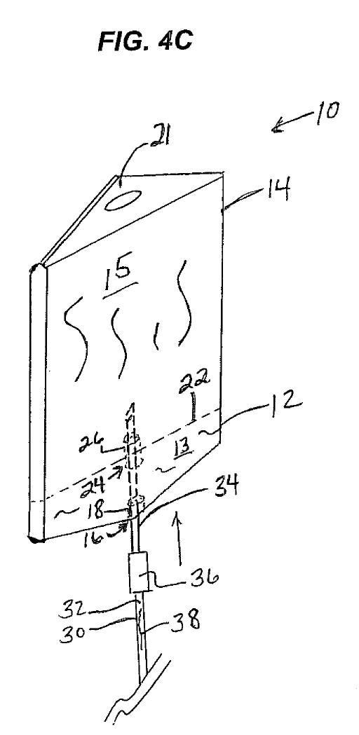

In another aspect the present invention provides an assembly for holding and delivering an intravenous fluid to an individual in need of such fluid (see FIG. 4a), The assembly may comprise a container 10 substantially as described above, including at least one first portion 12 for holding a purge fluid 13 and at least one second portion 14 for holding a medication 15, wherein the medication 15 and purge fluid 13 are not admixed. The assembly further includes a length of tubing 30. As described above, the at least one first portion 12 may be separated from the second portion 14 by at least one divider 22. In the depicted embodiment, first portion 12 is spatially oriented below second portion 14, allowing a gravity feed of purge fluid 13 and medication 15.

Divider 22 may include at least one access point 24 between the at least one first portion 12 and the second portion 14. Access point 24 may consist of a divider-traversing port 26 having a self-sealing septum 28 (see FIG. 1), or may be a self-sealing divider 22 functioning as a partition or wall (see FIG. 3), and including at least a self-sealing portion as described above. It will be appreciated that the at least one first portion 12 should hold a volume of purge fluid 13 at least sufficient to substantially purge a lumen 32 of the tubing 30 of air or any other fluid contained therein, but may be adapted to hold only a volume of purge fluid 13 sufficient to substantially purge lumen 32. Optionally, the tubing 30 may be provided with a needle or trocar 34 (see FIGS. 4A-4D) at opposed ends thereof, for piercing container 10 and the skin and blood vessel (not shown), respectively, of an individual receiving the contents of container 10.

The present invention further provides a method for delivering a medication into a blood vessel of an individual, comprising providing a container 10 having at least one first portion 12 containing a purge fluid 13 and a second portion 14 containing a medication 15. The invention further contemplates providing a length of tubing 32 defining a lumen 32, and placing the tubing lumen 32 in fluid communication with the at least one first portion 12 of the container 10.

As described above, prior to use the lumen 32 of tubing 30 (such as tubing for delivering fluids intravenously) typically contains a volume of a fluid 38, often a gas such as air. Delivery of such a fluid 38 into a blood vessel of an individual is undesirable. Accordingly, in the present method at least a sufficient volume of the purge fluid 13 is passed through the tubing lumen 32 to purge the volume of the fluid 38 contained in said lumen 32. The lumen 32 of the tubing 30 is then placed in fluid communication with the second portion 14 of the container.

The method of the present invention (depicted in FIGS. 4a-4c) may be accomplished by providing a hollow needle or trocar 34 at an end of the tubing 30. It is known in this art to provide such a needle or trocar 34 for tubing for intravenous delivery. The needle or trocar 34 may be integral to the tubing 30, or alternatively may be attached thereto by a suitable connector 36 (see FIGS. 4a-4c), such as a threaded connector, a friction or interference fit connector, the connector marketed in association with the trademark LUER-LOK, or the like.

First, the needle or trocar 34 may be advanced into the first portion 12 of container 10 (see FIG. 4b), such as through port 18. The user then allows a sufficient volume of purge fluid 13 to pass through the tubing lumen 32 to substantially displace the volume of fluid 38 contained therein. Next, the user may advance the needle or trocar 34 into the second portion 14, to allow delivery of the medication 15 contained therein. Of course, in the embodiment shown in FIG. 1 and FIGS. 4a-4c, the user would advance the needle or trocar 34 into second portion 14 via port 26. In the embodiment shown in FIG. 3, the user would simply puncture or pierce divider 22 using needle or trocar 34 to access the second portion 14 of container 10. Regardless, the skilled artisan will appreciate that the method of the present invention allows providing a purge fluid 13 and a medication 15 in the same container 10, and also purging a tubing lumen 32 of air or other gas using purge fluid 13 followed by delivery of medication 15, all without admixing purge fluid 13 and medication 15. Upon withdrawal of needle or trocar 34 from second portion 14, it will be appreciated that the self-sealing nature of port 26 (FIGS. 1, 4a-4c) or of divider 22 (FIG. 3) substantially prevents admixing of the contents of second portion 14 and first portion 12.

Still further (embodiment not shown), it will be apparent to the skilled artisan that in practicing the present method it would be possible to select a first fluid and a second fluid which are sufficiently immiscible that substantial admixing or emulsification of the two is not possible. For example, a medication, or a diluent or carrier for the medication, could be selected which is sufficiently immiscible with a selected purge fluid whereby, when placed in container 10, a divider 22 is defined by an interface between the first and second fluids 12, 14. In this fashion, separation of first and second fluids 12, 14 is made possible, while allowing access to second fluid 14 from first fluid 12, such as by passing a needle or trocar 34 therethrough as described above.

Thus, the risk of contamination of needle or trocar 34 during such a purging operation is substantially reduced by use of the container and method of the present invention. In contrast, conventional methods of purging a tubing lumen 32, such as by passing needle or trocar 34 into a first container containing a purge fluid, withdrawing needle or trocar 34 therefrom, and subsequently passing needle or trocar 34 into a second container holding a medication or other intravenous fluid, risks incidental contact of needle or trocar 34 with a non-sterile item such as the users band, the exterior of the second container, or the like.

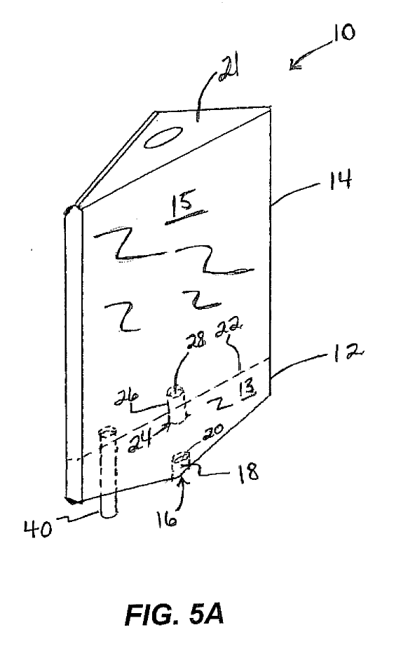

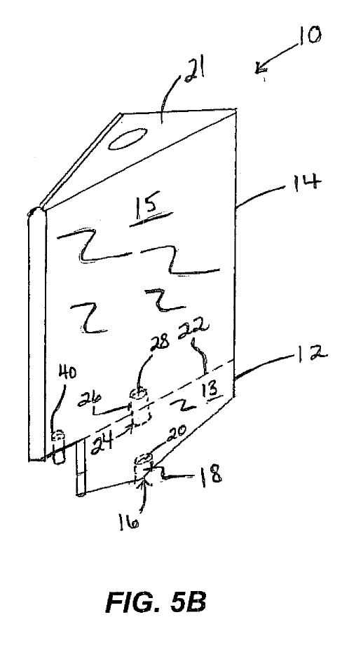

The foregoing description of a preferred embodiment of the invention has been presented for purposes of illustration and description. It is not intended to be exhaustive or to limit the invention to the precise form disclosed. Obvious modifications or variations are possible in light of the above teachings. For example, as shown in FIGS. 5a and 5b, a container 10 substantially as described above may be provided, having a separate injection cap or port 40 whereby a medication or ingredient may be injected or infused into second portion 14 of container 10 without requiring passage through first portion 12. In one embodiment a port 40 may be provided having a sufficient length to span a dimension of first portion 12 (see FIG. 5a). Alternatively a port 40 may be provided which does not pass through first portion 12 (see FIG. 5b).

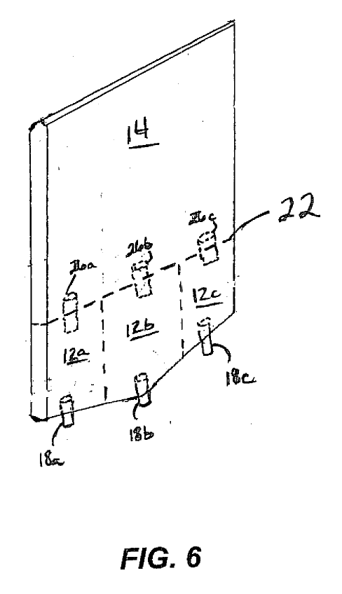

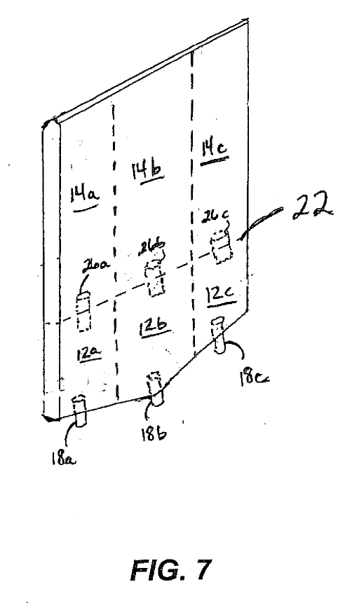

Still further, containers 10 having multiple first portions 12a-12c (see FIG. 6), or alternatively containers 10 having multiple first portions 12a-12c and multiple second portions 14a-14c (see FIG. 7), may be provided. In the depicted embodiments, ports 18a-18c provide access to the interior of first portions 12a-12c, and ports 26a-26c provide access to second portions 14a-14c. The skilled artisan will appreciate that these latter embodiments find utility in the instance where repeated purging operations are desired between delivery operations of a single medication, or where delivery of multiple different medications, each held in a separate second portion 14, from multiple intravenous lines (not shown) is desired.

The embodiment was chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the foregoing description and appended claims, when interpreted in accordance with the breadth to which they are fairly, legally and equitably entitled.

What is claimed is:

1. A container for holding an intravenous fluid, comprising:
at least one first portion for holding a purge fluid for purging air or other gas from a lumen of a tubing;
at least one second portion for holding a medication; and
at least one divider for separating the purge fluid and the medication and for providing access to an interior of the at least one second portion from an interior of the at least one first portion;
wherein the at least one divider prevents substantial admixing of the purge fluid and the medication, including when the interior of the at least one second portion is accessed from the interior of the at least one first portion.

2. The container of claim 1, wherein the at least one divider includes at least one self-sealing access point between the at least one first portion and the at least one second portion.

3. The container of claim 2, wherein the at least one self-sealing access point is fabricated from a suitable self-sealing material.

4. The container of claim 2, wherein the at least one self-sealing access point is at least one divider-traversing port having a self-sealing septum.

5. The assembly of claim 1, wherein the at least one first portion is spatially oriented beneath the at least one second portion with the at least one divider interposed therebetween.

6. An assembly for holding and delivering an intravenous fluid, comprising;
a container comprising at least one first portion for holding a purge fluid and at least one second portion for holding a medication, wherein the container further comprises at least one divider for separating the purge fluid and medication whereby the purge fluid and medication are not substantially admixed, including when an interior of the at least one second portion is accessed from an interior of the first portion; and
a length of tubing defining a lumen, wherein the tubing is adapted to place the lumen in fluid communication with the at least one first portion and the second portion.

7. The assembly of claim 6, wherein the at least one first portion is spatially oriented beneath the at least one second portion with the at least one divider interposed therebetween.

8. The assembly of claim 6, wherein the at least one divider includes at least one self-sealing access point between the at least one first portion and the at least one second portion.

9. The assembly of claim 8, wherein the at least one self-sealing access point is fabricated from a suitable self-sealing material.

10. The assembly of claim 8, wherein the at least one self-sealing access point is at least one divider-traversing port having a self-sealing septum.

11. The assembly of claim 6, wherein the at least one first portion holds a volume of fluid sufficient to substantially purge air or other fluid from a lumen of the tubing.

12. A method for delivering a medication into a blood vessel of an individual, comprising;
providing a container having at least one first portion containing a purge fluid and at least one second portion containing a medication wherein the container comprises at least one divider which prevents substantial admixing of the purge fluid and the medication, including when an interior of the at least one second portion is accessed from an interior of the first portion, further wherein the at least one first portion is spatially oriented below the at least one second portion with the divider interposed therebetween;
providing a length of tubing defining a lumen;
placing the tubing lumen in fluid communication with the at least one first portion and passing at least a sufficient volume of the purge fluid through the lumen to substantially purge air or other fluids therefrom;
and
placing the lumen in fluid communication with the at least one second portion to deliver a volume of the medication into the blood vessel.

13. The method of claim 12, further including the steps of:
providing a hollow needle or trocar at an end of the tubing;
advancing the needle or trocar into the at least one first portion;
allowing a sufficient volume of purge fluid to pass through the tubing lumen to substantially displace the volume of fluid contained therein; and
advancing the needle or trocar into the second portion through the divider.

14. The method of claim 12, including providing at least one divider including at least one self-sealing access point between the at least one first portion and the at least one second portion.

15. The method of claim 14, including providing at least one divider having at least one self-sealing access point fabricated from a suitable self-sealing material.

16. The method of claim 15, including providing at least one divider having at least one divider-traversing port including a self-sealing septum.

17. The method of claim 12, including providing a container having at least one first portion adapted for holding a volume of purge fluid only sufficient to purge the tubing lumen of air or other fluids.
